(19) United States
(12) Patent Application Publication
(10) Pub. No.: US 2004/0096472 A1
(43) Pub. Date: May 20, 2004

(54) Composition gelled with a dextrin ester

(76) Inventor: Florence Tournilhac, Paris (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/632,897

(22) Filed: Aug. 4, 2003

Related U.S. Application Data
(60) Provisional application No. 60/402,070, filed on Aug. 9, 2002.
(60) Provisional application No. 60/402,072, filed on Aug. 9, 2002.
(60) Provisional application No. 60/402,073, filed on Aug. 9, 2002.

(30) Foreign Application Priority Data
Aug. 2, 2002 (FR) 02 09887
Aug. 2, 2002 (FR) 02 09888
Aug. 2, 2002 (FR) 02 09889

(51) Int. Cl. A61K 31/715; A61K 7/00

(52) U.S. Cl. 424/401; 514/58

(57) ABSTRACT

A physiologically acceptable composition, such as a cosmetic composition, comprising a continuous liquid fatty phase, which is gelled or thickened with at least one fatty acid ester of dextrin with a degree of substitution of less than 2, wherein the at least one fatty acid ester of dextrin makes it possible to obtain, for example, a stick that does not exude, and which gives a glossy, non-migrating and long-lasting deposit over time when applied.

This application claims benefit of U.S. Provisional Application Nos. 60/402,070, 60/402,072, and 60/402,073, all filed Aug. 9, 2002.

Disclosed herein are cosmetic or pharmaceutical compositions that may be used for caring for and/or making up the skin, semi-mucous membranes, mucous membranes and/or integuments.

It is common practice to find a thickened fatty phase in cosmetic or dermatological products, for example, ointments, anhydrous exfoliant or care gels, and solid compositions, such as deodorants, balms and lipsticks.

It is moreover well-known practice to thicken oils with thickening polymers. Unfortunately, the known oil thickeners may have to be used in large amounts in order to obtain a gel of high viscosity. However, an excessive amount of thickener can give the composition insufficient properties when it is intended for cosmetics, for example, a sticky feel and a lack of slipperiness, these drawbacks possibly being very inconvenient, or even unacceptable.

Styrene/ethylene-propylene/styrene triblock polymers such as those described in U.S. Pat. No. 5,221,534, used in large amounts to thicken the oily phase of a foundation, may have poor cosmetic properties: for example, the makeup can be very sticky and thick during application, and can give the user a sensation of heaviness. In addition, these polymers may not make it possible to obtain a foundation that can be stable for two months at 45° C.: the product may release oil at the surface of the composition and can therefore no longer be uniform. The user may then have shake the composition well before using it. If the composition is not shaken or is poorly shaken, the application of this composition to the skin can leave an uncomfortable greasy sensation and the makeup obtained may not be uniform, traces of color possibly being visible on the skin. Moreover, an excessively thick foundation may be difficult to render satisfactorily uniform before the user applies it to the skin, thus making it difficult to obtain a uniform makeup result on the skin.

Disclosed herein are compositions comprising a continuous or external liquid fatty phase thickened or gelled with at least one dextrin ester.

Dextrin esters are generally known as emulsion stabilizers and as oil thickeners in the prior art.

For example, U.S. Pat. No. 4,780,145 describes an anhydrous composition comprising a cyclic polydimethylsiloxane, a dextrin ester with a fatty acid and petroleum jelly or a plant oil. The said document illustrates the ability of dextrin esters to gel both polar and a polar oils.

Similarly, Japanese Patent Application No. 63 216 817 describes a gel comprising a cyclic silicone of low molecular weight, a fatty phase, i.e., petroleum jelly or lanolin, and a dextrin ester.

Japanese Patent Application No. 04 149 116 describes a transparent composition comprising a dextrin ester—such that its degree of polymerization ranges from 3 to 100 and the degree of substitution ranges from 1.4 to 2.4—and liquid paraffin.

International Patent Application No. 97/11678 describes an antiperspirant gel gelled, for example, with a dextrin ester. The dextrin ester described has a degree of esterification of greater than 2. This composition, which is presented as being stable, can have the drawback of exuding.

French Patent Application No. 0 008 157 relates to aqueous emulsions stabilized with a combination of a dextrin ester and a hydrophilic gelling agent.

U.S. Pat. No. 5,840,883 describes dextrin esters with a degree of substitution ranging from 1 to 3, for example, from 1.2 to 2.8, which can have improved gelling properties.

The present inventor has observed that the compositions of the prior art may have a tendency to become covered with fatty substance droplets over time, and the higher the temperature and/or humid conditions the quicker these compositions become covered with these droplets. This phenomenon of exudation of fatty substances can be very detrimental because it can put off users.

Thus, one aim of the present invention is to provide a composition thickened or gelled with at least one dextrin ester that can overcome at least one disadvantage of the prior art including not exuding, not releasing oil at the surface of the composition, and that is stable. Another aim is to provide a composition of uniform color that has at least one of these advantages. Yet another aim is to provide a composition that can give a glossy deposit on the skin and/or the lips. Another aim is to provide a composition that can give a comfortable film deposited on a keratin material.

The present inventor has surprisingly found that the use at least one dextrin ester can make it possible to thicken or even to gel certain liquid fatty phases without observing exude and/or release oil at the surface of the composition. Further, when the composition obtained is applied to the lips, it can give a glossy, non-migrating, comfortable, long-lasting film of uniform color that may not exude.

For example, the present inventor has found that the use, in the composition, of at least one fatty acid ester of dextrin with a degree of esterification of less than 2 and which shows affinity for the the at least one oil of the liquid fatty phase, may make it possible to avoid any exudation and to obtain a composition that has at least one of the abovementioned advantages.

Thus, disclosed herein are compositions comprising a continuous liquid fatty phase, thickened or gelled with at least one fatty acid ester of dextrin with a degree of substitution of less than 2. This at least one fatty acid ester of dextrin can have the advantage of retaining the oils comprising the fatty phase without making the composition matte, unlike waxes. It can also make it possible to obtain a composition that may not exude.

The present inventor has also surprisingly found that the use of at least one fatty acid ester of dextrin can make it possible to thicken or even to gel anhydrous compositions, which may not exude, without the need to use waxes. When the composition obtained is applied to the lips, it can give a glossy, non-migrating, comfortable film of uniform color that may not exude.

For example, the inventor has found that the use, in a wax-free anhydrous composition, of at least fatty acid ester of dextrin with a degree of esterification of less than 2, can make it possible to avoid any exudation and to obtain a composition that has at least one of these advantages.

This at least one fatty acid ester of dextrin can have the advantage of retaining the oils comprising the fatty phase without making the composition matte, unlike waxes. It can also make it possible to obtain a composition of improved stability.

The inventor has also surprisingly found that the use of a mixture of at least one fatty acid ester of dextrin with a degree of esterification of less than 2, and of at least one fatty acid ester of dextrin with a degree of substitution of greater than 2 can make it possible to obtain thickened or gelled compositions that may be stable over time. Furthermore, it may also be possible to obtain a transparent or translucent composition. When the composition obtained is applied to the lips, it can give a glossy, non-migrating, comfortable film of uniform color.

This mixture of the at least one dextrin esters can have at least one advantage of retaining the at least one oil comprising the fatty phase while also making it possible to obtain a composition with improved stability and/or with improved shine.

The compositions disclosed herein applies not only to makeup products for the lips, but also to care and/or treatment products for the skin, including the scalp, and the lips, such as antisun products, for example, in stick form; for facial skin, skin makeup products, for both the human face and body, such as foundations, for example, products cast in stick or in dish form; concealer products; eye-shadows and transfer tattoo products; body hygiene products such as deodorants, for example, in stick form; shampoos and conditioners; eye makeup products, such as eyeliners, for example, in pencil form and mascaras, for example, in the form of cake; and body and facial care products.

As used herein, the expression “dextrin” means a mixture of glucose, maltose and higher molecular weight saccharides formed by acid hydrolysis of starch. It is usually marketed as corn syrup. In one embodiment, dextrin can be used.

As used herein, the expression “liquid fatty phase” means a fatty phase that is liquid at room temperature (25° C.) and atmospheric pressure (760 mmHg), and comprises at least one fatty substance that is liquid at room temperature, also known as “oils”, which are generally mutually compatible.

As used herein, the expression “anhydrous cosmetic composition” means a composition to which water is not added during the formulation, such as a composition comprising a total content of water of not more than 2% by weight, relative to the total weight of the composition, such as not more than 0.5% by weight, relative to the total weight of the composition. In other words, the fatty outer or continuous phase represents up to 98% by weight of the composition, such as up to 99.5% by weight.

As used herein, the term “thickened” means a composition whose viscosity is increased by adding the at least one fatty acid ester of dextrin, and which flows under its own weight over time.

As used herein, the term “gelled” means a composition whose viscosity is increased by adding the at least one fatty acid ester of dextrin, and which does not flow under its own weight over time.

As used herein, the expression “transparent” or “translucent” means that a portion of visible light gets through a film of the composition disclosed herein, having a particular thickness.

When said portion of visible light diffuses, the composition is translucent and when said portion of visible light does not diffuse, the composition is transparent.

The translucent or transparent feature of the composition is determined as follows: the composition is eventually heated to be poured into a 30 ml Volga pot. The composition is made cold by exposure to ambient temperature for 24 hours.

A black cross with 2 nm thick lines is obtained on a white sheet of paper, and the pot filled with the composition is placed on top of the paper sheet.

If the cross can be seen at naked eye under daylight at a 40 cm maximum observation distance, then the composition is translucent or transparent.

The first embodiment disclosed herein is a composition comprising a continuous liquid fatty phase gelled or thickened with a sufficient amount of at least one fatty acid ester of dextrin with a degree of substitution of less than 2 on the basis of one repeating unit, for example one glucose unit, wherein the fatty phase comprises at least one oil having solubility parameters δd, δp and δh satisfying the following conditions:

[4(δd−δdₑ)²+(δp−δpₑ)²+(δh−δhₑ)²]^{1/2}≦10.

wherein δdₑ, δpₑ and δhₑ are the solubility parameters of the at least one fatty acid ester of dextrin, and wherein the liquid fatty phase and the at least one fatty acid ester of dextrin form a physiologically acceptable medium.

The second embodiment disclosed herein is thus a wax-free anhydrous composition comprising a continuous liquid fatty phase thickened or gelled with a sufficient amount of at least one fatty acid ester of dextrin with a degree of substitution of less than 2 on the basis of one repeating unit, for example one glucose unit, wherein the liquid fatty phase and the at least one fatty acid ester of dextrin form a physiologically acceptable medium.

The third embodiment disclosed herein is a composition comprising a continuous liquid fatty phase gelled or thickened with a sufficient amount of a mixture comprising at least one fatty acid ester of dextrin with a degree of substitution of less than 2 on the basis of one repeating unit, for example one glucose unit, and at least one fatty acid ester of dextrin with a degree of substitution of greater than 2 on the basis of one repeating unit, for example one glucose unit, wherein the liquid fatty phase and the at least one dextrin ester form a physiologically acceptable medium.

Dextrin Ester

According to any one embodiment, the at least one fatty acid ester of dextrin with a degree of substitution of less than 2 on the basis of one repeating unit, for example one glucose unit, corresponds to formula (I) below:

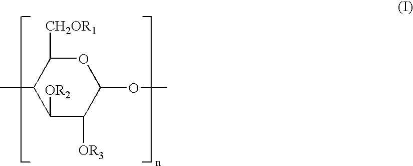

wherein:

the radicals R₁, R₂ and R₃, which may be identical or different, are each chosen from hydrogen and acyl groups (R—CO—), wherein the radical R is chosen from linear and branched, saturated and unsaturated hydrocarbon-based groups comprising from 6 to 50 carbon atoms, for example, 8 to 30 carbon atoms, further, for example, from 12 to 22 carbon atoms, and even further, for example, from 12 to 18 carbon atoms, with the proviso that at least one of the radicals R₁, R₂ or R₃ is not a hydrogen atom, and

n is an integer ranging from 3 to 150, for example, from 10 to 100, even further, for example, from 15 to 40.

According to another embodiment, the at least one fatty acid ester of dextrin with a degree of substitution of less than 2 on the basis of one repeating unit, for example one glucose unit, corresponds to formula (II):

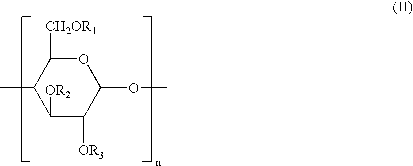

wherein:

the radicals R₁, R₂ and R₃, which may be identical or different, are each chosen from hydrogen and acyl groups (R—CO—), wherein the radical R is chosen from linear and branched, saturated and unsaturated hydrocarbon-based groups comprising 6 to 50 carbon atoms, for example, 8 to 30 carbon atoms, further, for example, from 12 to 22 carbon atoms, and even further, for example, from 12 to 18 carbon atoms, with the proviso that at least one of the radicals R₁, R₂ or R₃ is not a hydrogen atom, and

n is an integer ranging from 3 to 150, for example, from 10 to 100, and further, for example, from 15 to 40.

The acyl group R—CO— of the at least one fatty acid ester of dextrin of formula (I) or (II) may, for example, be chosen from at least one of caprylyl, caproyl, lauroyl, myristyl, palmityl, stearyl, eicosanyl, docosanoyl, isovaleryl, 2-ethylbutyryl, ethylmethylacetyl, isoheptanyl, 2-ethylhexanyl, isononanyl, isodecanyl, isotridecanyl, isomyristyl, isopalmityl, isostearyl, isohexanyl, decenyl, dodecenyl, tetradecenyl, myristyl, hexadecenoyl, palmitoleyl, oleyl, elaidyl, eicosenyl, sorbyl, linoleyl, linolenyl, punicyl, arachidonyl and stearoyl radicals.

The acyl group R—CO may, for example, be linear. The acyl group R—CO may, for example, be a palmitic radical. For example, the at least one fatty acid ester of dextrin palmitate can be used in the compositions disclosed herein, such that the degree of substitution is less than 1.9, for example, less than 1.8, and further, for example, ranging from 1.5 to 1.7. The integer n can range, for example, from 25 to 35, further, for example, from 27 to 33 and even further, for example, can be equal to 30.

The weight-average molecular weight of the at least one fatty acid ester of dextrin with a degree of substitution of less than 2 can range, for example, from 10 000 to 30 000 and further, for example, from 15 000 to 20 000. The weight-average molecular weight is determined by gas chromatography, with a polystyrene standard.

Some of the at least one fatty acid esters of dextrin are commercially available, for example, under the name Rheopearl TL from the company Chiba Flour.

The at least one fatty acid ester of dextrin with a degree of substitution of less than 2 on the basis of one repeating unit, for example one glucose unit, may be present in the composition disclosed herein in an amount ranging from 1% to 50% by weight, relative to the total weight of the composition, for example, from 4% to 30% by weight, relative to the total weight of the composition, further, for example, from 4% to 25% by weight, relative to the total weight of the composition, further, for example, from 5% to 25% by weight, relative to the total weight of the composition, further, from 4% to 25% by weight, relative to the total weight of the composition, and even further, for example, from 10% to 25% by weight, relative to the total weight of the composition.

The at least one fatty acid ester of dextrin with a degree of substitution of greater than 2 on the basis of one repeating unit, for example one glucose unit, may be present in the composition disclosed herein in an amount ranging from 1% to 50% by weight, relative to the total weight of the composition, for example, from 4% to 30% by weight, relative to the total weight of the composition, further, for example, from 4% to 25% by weight, relative to the total weight of the composition, further, for example, from 5% to 25% by weight, relative to the total weight of the composition, further, for example, from 4% to 25% by weight, relative to the total weight of the composition, and even further, for example, from 10% to 25% by weight, relative to the total weight of the composition.

The thickening or gelation of the at least one oil of the liquid fatty phase, which may be partly modified by the nature and amount of the at least one fatty acid ester of dextrin used, is such that a rigid structure is obtained, in the form of a tube or a stick, or in pasty form. The tubes, when they are colored, can make it possible after application to obtain a glossy deposit that is uniform in color and that does not migrate in the wrinkles and fine lines of the skin, for example, surrounding the lips, but also the eyes.

As used herein, the thickening or gelation of the liquid fatty phase is obtained with the aid of at least one fatty acid ester of dextrin with a degree of substitution of less than 2 on the basis of one repeating unit, for example one glucose unit, for example at least one fatty acid ester of dextrin of formula (I) or (II).

The at least one fatty acid ester of dextrin with a degree of substitution of greater than 2 on the basis of one repeating unit, for example one glucose unit, corresponding to formula (III) below can also be used in the composition disclosed herein:

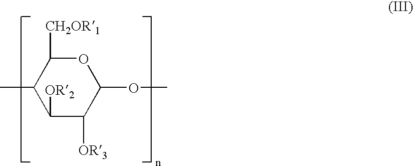

wherein:

the radicals R′₁, R′₂ and R′₃, which may be identical or different, are each chosen from hydrogen and acyl groups (R′—CO—), wherein the radical R′ is chosen from linear and branched, saturated and unsaturated hydrocarbon-based groups comprising from 6 to 50 carbon atoms, for example, from 8 to 30 carbon atoms, even further, for example, from 12 to 22 carbon atoms, and further, for example, from 12 to 18 carbon atoms, with the proviso that at least one of the radicals R′₁, R′₂ or R′₃ is not a hydrogen atom, and

n is an integer ranging from 3 to 150, for example, from 10 to 100, and further, for example, from 15 to 40;

R′ and n may have the same meaning of R and n described previously.

For example, at least one fatty acid ester of dextrin with a degree of substitution of greater than 2 on the basis of one repeating unit, for example one glucose unit, is used such that the degree of substitution is greater than 2.1, for example, ranging from 2.1 to 2.3.

The weight-average molecular weight of the fatty acid ester of dextrin with a degree of substitution of greater than 2 on the basis of one repeating unit, for example one glucose unit, ranges, for example, from 10 000 to 30 000 and further, for example, from 15 000 to 20 000. The weight-average molecular weight is determined by gas chromatography with a polystyrene standard.

Examples of the at least one fatty acid ester of dextrin of formula (III) that may be used in the composition disclosed herein include Rheopearl KL sold by the company Chiba Flour.

The mixture comprising at least one fatty acid ester of dextrin with a degree of substitution of less than 2 on the basis of one repeating unit and at least one fatty acid ester of dextrin with a degree of substitution of greater than 2 on the basis of one repeating unit may, for example, be present in the composition disclosed herein in an amount, for example, of less than or equal to 50% by weight, relative to the total weight of the composition, for example, ranging from 5% to 50% by weight, relative to the total weight of the composition, further, for example, from 5% to 40% by weight, relative to the total weight of the composition, and further, for example, from 5% to 25% by weight, relative to the total weight of the composition, and even further, for example, from 9% to 25% by weight, relative to the total weight of the composition.

The at least one fatty acid ester of dextrin with a degree of substitution of less than 2 and the at least one fatty acid ester of dextrin with a degree of substitution of greater than 2 may, for example, be present in relative mass proportions ranging from 1:10 to 3:1, for example, from 1:8 to 3:2, further, for example, from 1:2 to 3:2, even further, for example, from 0.8:1 to 1.2:1, further, for example, from 0.9:1 to 1.1:1, and even further, for example, from 1:2 to 1:1.

Fatty Phase

According to the embodiments disclosed herein, the liquid fatty phase of the composition may comprise a volatile liquid organic phase.

According to the second and the third embodiments disclosed herein, the liquid fatty phase of the composition may also comprise a non-volatile liquid organic phase.

According to the first embodiment disclosed herein, the liquid fatty phase of the composition may also comprise at least one non-volatile liquid organic oil different from the at least one oil showing specific solubility parameters.

The expression “volatile organic phase” means any non-aqueous medium capable of evaporating from the skin in less than one hour at room temperature and atmospheric pressure. This volatile phase may, for example, comprise oils with a vapor pressure, at room temperature (25° C.) and atmospheric pressure (760 mm Hg), ranging from 0.13 Pa to 40 000 Pa (10⁻³ to 300 mm Hg), for example, ranging from 1.3 Pa to 13 000 Pa (0.1 to 100 mm Hg) and further, for example, ranging from 1.3 Pa to 13 000 Pa (0.01 to 10 mm Hg).

The expression “non-volatile organic phase” means any medium capable of remaining on the skin for several hours. A non-volatile liquid organic phase, for example, has a non-zero vapor pressure at room temperature and atmospheric pressure, of less than 0.001 mm Hg (0.13 Pa).

The liquid fatty phase may comprise at least one oil chosen, for example, from carbon-based oils, hydrocarbon-based oils, fluoro oils and silicone oils of mineral, animal, plant and synthetic origin, provided that they form a uniform and macroscopically stable mixture and that they are suitable for the intended use.

The term “hydrocarbon-based oil” means an oil formed from, or comprising, carbon and hydrogen atoms, and optionally oxygen and nitrogen atoms, and comprising no silicone or fluorine atoms. It may also comprise at least one functional group chosen from alcohol, ester, ether, carboxylic acid, amine and amide functional groups.

The total liquid fatty phase of the composition, i.e., the total amount of the at least one oil, can be present in an amount ranging, for example, from 5% to 95% by weight, relative to the total weight of the composition, further, for example, from 20% to 75% by weight, relative to the total weight of the composition, and even further, for example, from 30% to 70% by weight, relative to the total weight of the composition.

Further, for example, the liquid fatty phase may comprise at least one volatile organic oil chosen from oils that are volatile at room temperature, such as volatile cosmetic oils. The at least one volatile organic oil is favorable for obtaining a transfer-resistant deposit with good staying power. It can also facilitate the application of the composition to the skin. The at least one volatile organic oil may, for example, be chosen from hydrocarbon-based oils, silicone oils and fluoro oils and may optionally comprise at least one group chosen from alkyl and alkoxy groups, pendent or at the end of the silicone chain.

The at least one volatile organic oil that may be used in the compositions disclosed herein may, for example, be chosen from:

linear and cyclic silicone oils with a viscosity at room temperature of less than 8 mm²/s and, for example, comprising from 2 to 7 silicone atoms, these silicones optionally comprising at least one group chosen from alkyl and alkoxy groups comprising from 1 to 10 carbon atoms. The volatile silicone oils that may be used in the compositions disclosed herein may, for example, be chosen from octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and

hydrocarbon-based volatile oils comprising from 8 to 16 carbon atoms, for example, at least one of C₈-C₁₆ branched alkanes, such as C₈-C₁₆ isoalkanes (also known as isoparaffins), isododecane, isodecane, isohexadecane and, for example, the oils sold under the trade-names “Isopar” and “Permethyl”, branched C₈-C₁₆ esters, such as isohexyl neopentanoate; isododecane may, for example, be used.

For example, the at least one volatile organic oil may be present in amount ranging, for example, from 0.1% to 80% by weight, relative to the total weight of the composition, further, for example, from 1% to 60% by weight, relative to the total weight of the composition, and even further, for example, from 5% to 50% by weight, relative to the total weight of the composition.

The composition disclosed herein may also comprise at least one non-volatile oil. The at least one non-volatile oil that may be used in the compositions disclosed herein may, for example, be chosen from hydrocarbon-based oils of mineral and synthetic origin, such as linear and branched hydrocarbons, for example, liquid paraffin and derivatives thereof, liquid petroleum jelly, polydecenes, hydrogenated polyisobutene such as Parleam sold by the company Nippon Oil Fats, squalane of synthetic and plant origin; oils of animal origin, such as mink oil, turtle oil, perhydrosqualene; hydrocarbon-based oils of plant origin with a high triglyceride content, comprising fatty acid esters of glycerol, the fatty acids of which may comprise at least one fatty chain chosen from fatty chains having varied chain lengths, and the at least one fatty chain possibly being chosen from linear and branched, and saturated and unsaturated fatty chains, for example, fatty acid triglycerides, for example, fatty acid triglycerides comprising from 4 to 22 carbon atoms, such as the triglycerides of heptanoic and octanoic acid, and of capric/caprylic acid, and hydroxylated triglycerides, such as sweet almond oil, beauty-leaf oil, palm oil, grapeseed oil, sesame seed oil, arara oil, rapeseed oil, sunflower oil, cotton seed oil, apricot oil, castor oil, alfafa oil, marrow oil, blackcurrant oil, macadamia oil, musk rose oil, hazelnut oil, avocado oil, jojoba oil, olive oil, cereal (maize, wheat, barley and rye) germ oil and karite butter; fatty acid esters, for example, comprising from 4 to 22 carbon atoms, such as, fatty acid esters of octanoic acid, of heptanoic acid, of lanolic acid, of oleic acid, of lauric acid and of stearic acid, such as propylene glycol dioctanoate, propylene glycol monoisostearate, polyglyceryl-2 diisostearate and neopentyl glycol diheptanoate; synthetic esters of formula R₁COOR₂ wherein R₁ is chosen from linear and branched higher fatty acid residues comprising from 7 to 40 carbon atoms and R₂ is chosen from branched hydrocarbon-based chains comprising from 3 to 40 carbon atoms, for example, purcellin oil (cetostearyl octanoate), isononyl isononanoate, C₁₂ to C₁₅ alkyl benzoate, 2-ethylhexyl palmitate, 2-octyldodecyl stearate, 2-octyldodecyl erucate, isostearyl isostearate, 2-octyldodecyl benzoate, alcohol and polyalcohol octanoates, decanoates and ricinoleates, isopropyl myristate, isopropyl palmitate, butyl stearate, hexyl laurate, diisopropyl adipate, 2-ethylhexyl palmitate, 2-hexyldecyl laurate, 2-octyldecyl palmitate, 2-octyldodecyl myristate, 2-diethylhexyl succinate, diisostearyl malate and isodecyl neopentanoate; hydroxylated esters, for example, isostearyl lactate, octyl hydroxystearate, octyldodecyl hydroxystearate, diisostearyl malate, triisocetyl citrate, glyceryl triisostearate and diglyceryl triisostearate; diethylene glycol diisononanoate; pentaerythritol esters; esters of aromatic acids and of alcohols comprising 4 to 22 carbon atoms, for example, tridecyl trimellitate; C₈-C₂₆ higher fatty acids such as oleic acid, linoleic acid, linolenic acid and isostearic acid; C₈-C₂₆ higher fatty alcohols such as oleyl alcohol, linoleyl alcohol, linolenyl alcohol, isostearyl alcohol and octyldodecanol; synthetic esters comprising at least 7 carbon atoms, silicone oils such as linear polydimethylsiloxanes (PDMS) that are liquid at room temperature, and optionally phenylated, such as phenyltrimethicones, phenyltrimethylsiloxydiphenylsiloxanes, diphenyl dimethicones, diphenyl methyldiphenyl trisiloxanes, 2-phenylethyl trimethylsiloxysilicates that are liquid, optionally substituted with at least one group chosen from aliphatic and aromatic groups, for example, alkyl, alkoxy and phenyl groups, pendent and/or at the end of a silicone chain, the at least one group comprising from 2 to 24 carbon atoms and optionally substituted with at least one fluoro group, and/or with at least one functional group chosen from hydroxyl, thiol and amine functional groups; polysiloxanes modified with fatty acids, with fatty alcohols and with polyoxyalkylenes, for example, dimethicone copolyols and alkylmethicone copolyols; and liquid fluorosilicone.

The composition may comprise at least one non-volatile oil present in an amount ranging, for example, from 0.1% to 80% by weight, relative to the total weight of the composition, further, for example, ranging from 1% to 60% by weight, relative to the total weight of the composition, and even further, for example, ranging from 5% to 50% by weight, relative to the total weight of the composition.

According to the first embodiment disclosed herein, the composition comprises a continuous liquid fatty phase gelled or thickened with a sufficient amount of at least one fatty acid ester of dextrin with a degree of substitution of less than 2 on the basis of one repeating unit, for example one glucose unit, the said fatty phase comprising at least one oil that has solubility parameters δd, δp and δh satisfying the following conditions:

[4(δd−δdₑ)²+(δp−δpₑ)²+(δh−δhₑ)²]^{1/2}≦10.

δdₑ, δpₑ and δhₑ being the solubility parameters of the at least one fatty acid ester of dextrin, and wherein the liquid fatty phase and the at least one fatty acid ester of dextrin form a physiologically acceptable medium.

The definition and calculation of the solubility parameters in the three-dimensional Hansen solubility space are described in the article by C. M. Hansen: “The three dimensional solubility parameters” J. Paint Technol. 39, 105 (1967).

The three Hansen solubility parameters —δd, δp and δh—characterize, for a given constituent, the energies corresponding, respectively, to the dispersive interactions, polar interactions and interactions of hydrogen bonding type existing between the molecules of this constituent.

In order for the at least one oil of the liquid fatty phase to be compatible with the at least one fatty acid ester of dextrin used, the solubility parameters of the at least one oil is chosen so as to be within the solubility sphere of the at least one fatty acid ester of dextrin, defined by a center of coordinates δdₑ, δpₑ and δhₑ and a radius R of not more than 10. The radius is calculated according to the following formula:

R=[4(δd−δdₑ)²+(δp−δpₑ)²+(δh−δhₑ)²]^{1/2}.

According to any one embodiment, R is less than or equal to 8, for example, equal to 5.

The values of the Hansen solubility parameters for the at least one fatty acid ester of dextrin are, for example, such that 17≦δdₑ≦19, 1≦δpₑ≦2, and 9≦δhₑ≦11.

The values of the Hansen solubility parameters for Rheopearl TL are, respectively, δdₑ=18.2, δpₑ=1.5 and δhₑ=9.8.

The liquid fatty phase may comprise a mixture of oils. In this case, the solubility parameters of the mixture are determined from those of the oils taken separately, according to the following relationships:

δ_{Dmixt}=Σᵢxiδ_{DI} δₚₘᵢₓₜ=Σᵢxiδₚᵢ and δₕₘᵢₓₜ=Σᵢxiδδₕᵢ

wherein xi represents the volume fraction of the oil (i) in the mixture.

The at least oil, alone, that satisfies these conditions may, for example, be chosen from:

fatty acid triglycerides, for example, comprising from 4 to 22 carbon atoms, such as heptanoic, octanoic and capric/caprylic acid triglycerides,

hydroxylated triglycerides, for example, castor oil,

synthetic alcohols comprising from 12 to 26 carbon atoms, such as octyidodecanol, 2-butyloctanol, 2-hexyldecanol and 2-undecylpentadecanol,

fatty acid esters, for example, comprising from 4 to 22 carbon atoms, such as propylene glycol dioctanoate, propylene glycol monoisostearate, neopentyl glycol diheptanoate, diethylene glycol diisononanoate; polyglyceryl 2 diisostearate, and oleyl alcohol,

hydroxylated synthetic esters, for example, isostearyl lactate, octyl hydroxystearate, octyldodecyl hydroxystearate, diisostearyl malate and triisocetyl citrate, and

esters of aromatic acids and of alcohols comprising from 4 to 22 carbon atoms, such as tridecyl trimellitate.

Table 1 below gives the value of the radius of the sphere R=[4(δd−18.2)²+(δp−1.5)²+(δh−9.8)²]^{1/2} for various oils relative to the values of the Hansen solubility parameters for Rheopearl TL. The following oils can be used in the compositions disclosed herein, without limiting the scope thereof. Other oils that satisfy the conditions disclosed above are available.

TABLE 1

| | | | | Distance |
|---|---|---|---|---|
| | | | | between the oil |
| | | | | and Rheopearl |
| Name of the oil | δd | δp | δh | TL |
| Castor oil | 16.79 | 1.27 | 9 | 2.9 |
| 2-Octyldodecanol | 16.36 | 1.43 | 7.56 | 4.3 |
| Oleyl alcohol | 16.28 | 1.6 | 8.01 | 4.2 |
| Caprylic/capric acid triglyceride | 16.64 | 1.69 | 6.47 | 4.6 |
| 2-Ethylhexyl glyceryl ether | | | | |
| palmitate | 16.53 | 1.71 | 7.98 | 3.8 |
| Hexyldecanol | 16.26 | 1.75 | 8.37 | 4.1 |
| 2-Ethylhexyl glyceryl behenate | 16.57 | 1.42 | 7.27 | 4.1 |
| Propylene glycol | | | | |
| monoisostearate | 16.36 | 1.89 | 8.53 | 3.9 |
| Pentaerythrityl tetraethyl-2- | 16.55 | 1.53 | 6.62 | 4.6 |
| hexanoate | | | | |
| Polyglyceryl 3 diisostearate | 16.96 | 1.64 | 10.27 | 2.5 |
| Isostearyl alcohol | 16.32 | 1.58 | 7.94 | 4.2 |
| Phytanetriol | 16.5 | 2.52 | 13.2 | 4.9 |
| Triisocetyl citrate | 16.77 | 1.09 | 6.74 | 4.2 |
| Triisoarachidyl citrate | 16.77 | 0.9 | 6.12 | 4.7 |
| Diisostearyl malate | 16.61 | 1.26 | 7.08 | 4.2 |
| Triisostearyl citrate | 16.77 | 0.99 | 6.4 | 4.5 |
| Oxyethylene (7 OE) glyceryl | 16.97 | 2.98 | 9.61 | 2.9 |
| triacetate | | | | |
| Tridecyl trimellitate | 17.49 | 1.12 | 5.23 | 4.8 |
| Glyceryl triheptanoate | 16.62 | 1.99 | 7.01 | 4.2 |
| Polyglyceryl-2 triisostearate | 16.7 | 1.06 | 6.61 | 4.4 |
| Undecylpentadecanol | 16.45 | 1.12 | 6.69 | 4.7 |
| PPG10 butanediol | 16.77 | 2.38 | 10.73 | 3.1 |
| Glyceryl triacetate | 16.42 | 4.57 | 10.63 | 4.8 |
| Octyl hydroxystearate | 16.43 | 1.55 | 7.73 | 4.1 |
| C12-13 alkyl lactate | 16.25 | 2.47 | 9.76 | 4.0 |
| Isostearyl lactate | 16.36 | 1.89 | 8.53 | 3.9 |
| 2-Octyldodecyl hydroxystearate | 16.53 | 1.09 | 6.47 | 4.7 |
| Butyl isostearate | 16.6 | 1.21 | 6.72 | 4.5 |
| Pentaerythrityl tetraisononanoate | 16.39 | 1.4 | 6.32 | 5.0 |
| Dipropylene glycol dibenzoate | 18.77 | 2.57 | 7.12 | 3.1 |
| Pentaerythrityl tetrapelargonate | 16.82 | 1.4 | 6.32 | 4.4 |
| Polyglyceryl-2 isostearate | 17.03 | 2.59 | 12.99 | 4.1 |
| Polyglyceryl-2 diisostearate | 16.79 | 1.5 | 8.95 | 2.9 |
| Glyceryl diisostearate | 16.61 | 1.29 | 7.16 | 4.1 |
| Triisodecyl trimellitate | 17.43 | 1.37 | 5.8 | 4.3 |
| Tris(2-ethylhexyl)trimellitate | 17.56 | 1.62 | 6.31 | 3.7 |
| Isofol-12 trimellitate | 17.34 | 1.19 | 5.4 | 4.7 |
| Glyceryl trioctanoate | 16.35 | 1.78 | 6.64 | 4.9 |
| 2-Butyloctanol | 16.12 | 2.26 | 9.52 | 4.2 |

Mixtures of the oils described above may be made with oils that are not found alone in the solubility sphere of the at least one fatty acid ester of dextrin as defined above, provided that the mixture is homogeneous and that the solubility parameters of the mixture of oils satisfy the conditions disclosed herein. It is within the scope of a person skilled in the art to determine the amounts of each oil to obtain a mixture of oils that satisfies the above conditions.

The following oils do not satisfy the above Hansen relationships, taken individually:

volatile and non-volatile silicones,

oils of mineral origin, such as liquid petroleum jelly and liquid paraffin,

oils of plant origin, such as jojoba oil, sesame oil, rapeseed oil and karite butter,

synthetic oils, such as Purcellin oil, 2-ethylhexyl palmitate, 2-octyidodecyl stearate, 2-octyldodecyl erucate, isostearyl isostearate, 2-octyidodecyl benzoate and hydrogenated isoparaffin (6.8 mol of isobutylene), synthetic oils and esters of formula R₅COOR₆ wherein R₅ is chosen from linear and branched higher fatty acid residues comprising from 1 to 40 carbon atoms, and further, for example, from 7 to 19 carbon atoms and R₆ is chosen from branched hydrocarbon-based chains comprising from 1 to 40 carbon atoms, and further, for example, from 3 to 20 carbon atoms, with R₅+R₆≧10, such as, for example, Purcellin oil (cetostearyl octanoate), isononyl isononanoate, C₁₂ to C₁₅ alkyl benzoate, isopropyl myristate, 2-ethylhexyl palmitate, and octanoates, decanoates and ricinoleates of alcohols and of polyalcohols.

The liquid fatty phase may, for example, be present in an amount ranging from 5% to 95% by weight, relative to the total weight of the composition, further, for example, from 20% to 75% by weight, relative to the total weight of the composition, and even further, for example, from 30% to 70% by weight, relative to the total weight of the composition.

The liquid fatty phase may also comprise at least one additional oil that is entirely incompatible with the oils described above, i.e., oils that do not form a mixture that is homogeneous to the eye and whose distance in the Hansen space is greater than 10 (in this case there will be no change in the solubility parameters of the initially optimized mixture). The at least one additional oil can, for example, be chosen from silicone oils such as volatile and non-volatile, linear and cyclic polydimethylsiloxanes (PDMS) that are liquid at room temperature; polydimethylsiloxanes comprising at least one group chosen from alkyl, alkoxy and phenyl groups, pendent and/or at the end of the silicone chain, the at least one group comprising from 2 to 24 carbon atoms; phenyl silicones, such as phenyl trimethicones, phenyl dimethicones, phenyl trimethylsiloxy diphenylsiloxanes, diphenyl dimethicones, diphenyl methyldiphenyl trisiloxanes and 2-phenylethyl trimethylsiloxysilicates; volatile and non-volatile, linear and branched fluorocarbons of synthetic and mineral origin; and polyglycols.

Other Ingredients

The composition disclosed herein may comprise at least one aqueous phase comprising water. The water may be chosen from at least one of floral water such as cornflower water, mineral water such as eau de Vittel, eau de Lucas and eau de La Roche Posay and spring water.

The aqueous phase may also comprise at least one organic solvent that is water-miscible (at 25° C.), for example, the at least one organic solvent may be chosen from primary alcohols such as ethanol and isopropanol, glycols such as glycerol, propylene glycol, butylene glycol, dipropylene glycol, diethylene glycol, glycol ethers, C₁ to C₄ alkyl ethers of monopropylene, dipropylene and tripropylene glycol and of monoethylene, diethylene and triethylene glycol.

The aqueous phase may also comprise at least one stabilizer, for example, the at least one stabilizer may be chosen from sodium chloride, magnesium dichloride and magnesium sulphate.

It may also be possible to incorporate any water-soluble or water-dispersible compound that is compatible with an aqueous phase, such as at least one of gelling agents, film-forming polymers, thickeners and surfactants.

According to one embodiment, the aqueous phase, for example, water, may be present in the composition disclosed herein in an amount ranging from 1% to 70% by weight, relative to the total weight of the composition, for example, from 5% to 50% by weight, relative to the total weight of the composition, and further, for example, from 5% to 20% by weight, relative to the total weight of the composition.

The composition disclosed herein may also comprise at least one additional additive usually used in the field under consideration. For example, the at least one additional additive may be chosen from antioxidants, preserving agents, neutralizers, lipophilic gelling agents and liquid non-aqueous compounds, aqueous-phase gelling agents, dispersing agents, and cosmetic active agents. The at least one additional additive may be present in the composition in an amount ranging, for example, from 0.0005% to 20% by weight, relative to the total weight of the composition, and further, for example, from 0.001% to 10% by weight, relative to the total weight of the composition.

The cosmetic active agents that may be used in the compositions disclosed herein may, for example, be chosen from at least one of vitamins A, E, C, B₃ and F, provitamins such as D-panthenol, calmant active agents, such as α-bisabolol, aloe vera, allantoin, plant extracts and essential oils, protecting agents and restructuring agents, such as ceramides, “refreshing” active agents, such as menthol and derivatives thereof, emollients (cocoa butter, dimethicone), moisturizers (arginine PCA), anti-wrinkle active agents, essential fatty acids and sunscreens.

Needless to say, a person skilled in the art will take care to select the optional additional additives and/or the amount thereof such that the advantageous properties of the composition disclosed herein are not, or are not substantially, adversely affected by the envisaged addition.

The compositions disclosed herein have numerous applications and concern all colored or uncolored cosmetic products and, for example, lipsticks and foundation.

The composition disclosed herein may be in the form chosen from colored and uncolored skincare compositions, antisun compositions, makeup-removing compositions and hygiene compositions. If the composition comprises cosmetic active agents, it may then be used as a care base and/or a non-therapeutic treatment base for the skin such as the hands and/or the face and/or for the lips (lip balms, for protecting the lips from cold and/or sunlight and/or wind) and/or an artificial tanning product for the skin.

The composition disclosed herein may also be in the form chosen from colored makeup products for the skin, for example, for the face, such as a blusher, a foundation, a makeup rouge and an eye-shadow; makeup products for the body, such as a semi-permanent tattoo product; makeup products for the lips, such as a lipstick and a lip gloss, optionally having care properties and/or non-therapeutic treatment properties; makeup products for the integuments, such as a nail varnish, a mascara and an eyeliner; and hair dye products and haircare products.

For example, according to one embodiment disclosed herein, the composition disclosed herein is in the form of a lipstick or a lip gloss. A glossy, non-migrating and long-lasting deposit may be produced when this lipstick is applied.

According to another embodiment disclosed herein, the composition is a foundation.

The composition disclosed herein may also comprise at least one pasty fatty substance and/or at least one wax.

As used herein, the term “pasty compound” means a compound with a melting point ranging, for example, from 25 to 60° C. and further, for example, from 30 to 45° C. and/or a hardness ranging, for example, from 0.001 to 0.5 MPa and further, for example, from 0.005 to 0.4 MPa.

As used herein, the term “wax” means a lipophilic fatty compound that is solid at room temperature (25° C.), with a reversible solid/liquid change of state, having a melting point of greater than 30° C., which may be up to 200° C., a hardness of greater than 0.5 MPa, and having an anisotropic crystal organization in the solid state. By bringing the wax to its melting point, it is possible to make it miscible with the oils and to form a microscopically homogeneous mixture, but on returning the temperature of the mixture to room temperature, recrystallization of the wax in the oils of the mixture is obtained.

For example, the physiologically-acceptable medium for the composition disclosed herein may comprise, besides the liquid fatty phase, at least one additional compound that may, for example, be chosen from fillers, waxes, oils, gums and pasty fatty substances, which are hydrocarbon-based, silicone-based and/or fluorinated, of plant, mineral and synthetic origin.

According to any one embodiment, the physiologically acceptable medium of the composition comprises at least one additional compound chosen from pasty fatty substances and waxes.

The waxes may, for example, be chosen from at least one of hydrocarbon-based waxes, silicone waxes, and fluoro waxes, optionally comprising at least one functional group chosen from ester and hydroxyl groups. For example, the waxes may be chosen from at least one of waxes of natural origin, such as optionally modified beeswax, carnauba wax, candelilla wax, ouricoury wax, Japan wax, cork fibre wax and sugarcane wax, ceresin, paraffin waxes, lignite waxes, microcrystalline waxes, lanolin wax, montan wax, ozokerites, hydrogenated oils, such as hydrogenated jojoba oil and products obtained by copolymerization of ethylene, the waxes obtained by Fischer-Tropsch synthesis, fatty acid esters and glycerides that are solid at 45° C., silicone waxes, such as alkyl and alkoxy poly(di)methylsiloxanes and poly(di)methylsiloxane esters that are solid at 45° C., comprising from 10 to 45 carbon atoms, and certain fatty acids, such as stearic acid, myristic acid and behenic acid.

For example, the composition disclosed herein comprises no more than 50% by weight, relative to the total weight of the composition, of waxes, further, for example, from 0.01% to 50% by weight, relative to the total weight of the composition, of waxes, further, for example, from 0.1% to 40% by weight, relative to the total weight of the composition, of waxes, and even further, for example, of no more than 30% by weight, relative to the total weight of the composition, of waxes, and further, for example, from 0.1% to 30% by weight, relative to the total weight of the composition, of waxes.

According to any one embodiment, the composition is free of waxes. A composition comprising 0.5% by weight or less than 0.5% by weight of waxes is considered as being free of waxes.

The nature and amount of the at least one additional compound chosen from pasty substances and waxes depend on the desired mechanical properties and textures. As a guide, the at least one additional compound chosen from pasty substances and waxes can, for example, be present in an amount ranging from 0.01% to 50% by weight, relative to the total weight of the composition, further, for example, from 0.1% to 40% by weight, relative to the total weight of the composition, further, for example, from 2% to 40% by weight, relative to the total weight of the composition, even further, for example, from 0.1% to 30% by weight, relative to the total weight of the composition, and further, for example, from 5% to 30% by weight, relative to the total weight of the composition.

According to any one embodiment, the physiologically acceptable medium of the composition comprises at least one additional gelling or thickening system, which results in a transparent or translucent phase.

Transparent waxes such as bis(1,1,1-trimethylolpropane) tetrastearate sold under the name Hest 2T-4S by the company Heterene may be used in the compositions disclosed herein, for example.

Moreover, the composition may comprise, at least one additional non-waxy gelling or thickening system. Said system can, for example, result in a transparent phase. The at least one additional non-waxy gelling or thickening system may, for example, be chosen from:

N-lauryl-L-glutamate α,γ-di-N-butylamide (LGBA) sold by the company Clariant and described in Zairyo Gijutsu, Vol. 10, No. 2, pp. 48-57 (1992)

monodibenzylidene sorbitol as described in U.S. Pat. No. 3,121,332, and

1,2- and 1,3-cyclohexane derivatives bearing at least one amide functional group as described in U.S. Pat. No. 6,410,003.

The expression “non-waxy system” means a gelling agent or thickener not corresponding to the definition of the waxes. A non-waxy system is a lipophilic fatty compound that is solid at room temperature (25° C.), which undergoes a reversible solid/liquid change of state, having a melting point of greater than 30° C. which may be up to 200° C., a hardness of greater than 0.5 MPa, and having in solid form an anisotropic crystal organization. By bringing a wax to its melting point, it is possible to make it oil-miscible and to form a microscopically homogeneous mixture, but on returning the temperature of the mixture to room temperature, recrystallization of the wax is obtained.

The term “filler” means any colorless particle chosen from lamellar, spherical and oblong mineral and organic fillers that are chemically inert in the composition. According to any one embodiment, the at least one filler may, for example, be less than 500 nm in size so as to preserve the transparent or translucent nature of the composition.

The at least one filler may, for example, be chosen from talc, mica, silica, kaolin, laponite, polyamide powders, such as Nylon® powder, poly-β-alanine powder and polyethylene powder, powders of tetrafluoroethylene polymers (Teflon®), lauroyllysine, starch, boron nitride, particles of acrylic polymer, for example, particles of acrylic acid copolymer, and silicone resin microbeads, precipitated calcium carbonate, dicalcium phosphate, magnesium carbonate, magnesium hydrocarbonate, hydroxyapatite. The at least one filler may or may not be surface-treated, for example, to make them lipophilic.

For example, the at least one filler may be present in an amount ranging, for example, from 0.1% to 60% by weight, relative to the total weight of the composition, further, for example, from 0.5% to 40% by weight, relative to the total weight of the composition, and even further, for example, from 1% to 35% by weight, relative to the total weight of the composition, and further, for example, from 1% to 25% by weight, relative to the total weight of the composition, if the at least one filler is present.

The term “physiologically or cosmetically acceptable” means having a pleasant taste, feel, appearance and/or odor, applicable ranging from several days to several months.

The composition disclosed herein may be manufactured by the known processes generally used in cosmetics.

The composition disclosed herein may, for example, comprise at least one dyestuff that may be chosen from lipophilic dyes, hydrophilic dyes, and pigments and nacres usually used in cosmetic and dermatological compositions. The at least one dyestuff may, for example, be present in an amount ranging, for example, from 0.01% to 40% by weight, relative to the total weight of the composition, further, for example, from 1% to 35% by weight, relative to the total weight of the composition, and even further, for example, from 5% to 25% by weight, relative to the total weight of the composition.

According to one embodiment disclosed herein, the at least one dyestuff may be chosen from dyes, pigments, and nacres so as to obtain a semi-covering or transparent makeup, i.e., a makeup that does not leave the skin, the lips or the integuments exposed. The pigments also can make it possible to reduce the sticky feel of the compositions, unlike soluble dyes.

The liposoluble dyes may, for example, be chosen from Sudan red, DC Red 17, DC Green 6, β-carotene, soybean oil, Sudan brown, DC Yellow 11, DC Violet 2, DC Orange 5 and quinoline yellow. The liposoluble dyes may be present in an amount ranging, for example, from 0.1 to 20% by weight, relative to the total weight of the composition, and further, for example, from 0.1% to 6% by weight, relative to the total weight of the composition (if present).

The pigments may, for example, be chosen from white and colored, mineral and organic, and coated and uncoated pigments. The mineral pigments may, for example, be chosen from titanium dioxide, optionally surface-treated, zirconium oxide, cerium oxide, iron oxide, chromium oxide, manganese violet, ultramarine blue, chromium hydrate and ferric blue. The organic pigments may, for example, be chosen from carbon black, pigments of D & C type, and lakes based on cochineal carmine and on barium, strontium, calcium and aluminium. The pigments may, for example, be present in an amount ranging, for example, from 0.1 to 40% by weight, relative to the total weight of the composition, further, for example, from 1 to 35% by weight, relative to the total weight of the composition, and even further, for example, from 2% to 25% by weight, relative to the total weight of the composition.

The nacreous pigments may, for example, be chosen from white nacreous pigments such as mica coated with titanium or with bismuth oxychloride, colored nacreous pigments such as titanium mica with iron oxides, titanium mica with, for example, ferric blue or chromium oxide, titanium mica with an organic pigment of the abovementioned type, and nacreous pigments based on bismuth oxychloride. The nacreous pigments may, for example, be present in an amount ranging from 0.1 to 20% by weight, relative to the total weight of the composition, further, for example, from 0.1% to 15% by weight, relative to the total weight of the composition.

For example, the composition disclosed herein may comprise a particulate phase present in an amount ranging, for example, from 5% to 70% by weight, relative to the total weight of the composition, further, for example, ranging from 10% to 60% by weight, relative to the total weight of the composition, and even further, for example, ranging from 15% to 40% by weight, relative to the total weight of the composition.

The particulate phase may comprise at least one particulate material chosen from the fillers, pigments and nacres as described above.

The composition disclosed herein may be manufactured by the known processes generally used in cosmetics or dermatology.

Forms of the Composition

The composition disclosed herein may be self-supporting and may be in the form of a stick or a dish. According to any one aspect, it is in the form of a transparent or transluscent rigid gel, for example, in the form of a transparent or transluscent stick, the liquid fatty phase forming the continuous phase. The composition can also be anhydrous.

The contents of the at least one fatty acid ester of dextrin of formula (I) or (II) may, for example, be chosen according to the desired hardness of the gel or stick and according to the particular intended application.

The hardness may be measured by the “cheese wire” method, which comprises cutting an 8.1 mm tube of lipstick and measuring the hardness at 20° C., using a DFGHS 2 tensile testing machine from the company Indelco-Chatillon pulling at a speed of 100 mm/minute. It is expressed as the shear force (expressed in grams) required to cut a stick under these conditions. According to this method, the hardness of a composition in stick form according to the composition disclosed herein ranges, for example, from 30 to 150 g, further, for example, from 30 to 120 and even further, for example, from 30 to 50 g.

This hardness is such that the composition is self-supporting and can disintegrate readily to form a satisfactory deposit on the skin and the lips. In addition, with this hardness, the composition disclosed herein in cast form, for example, cast as a stick, shows good impact strength.

According to the embodiments disclosed herein, the composition in stick form may have the behavior of a deformable and flexible elastic solid, and can give on application noteworthy elastic softness. The prior art compositions in stick form do not have this property of elasticity and flexibility.

When these sticks or tubes are colored, for example, pigmented, they can give on application a glossy deposit of uniform color that does not migrate in the wrinkles and fine lines of the skin, for example, surrounding the lips, but also the eyes.

According to any one embodiment, the composition is in the form of a care, cleansing, makeup-removing or makeup composition for the skin, semi-mucous membranes, mucous membranes and/or integuments.

The composition disclosed herein may, for example, be in a form chosen from pastes, solids and creams; oil-in-water and water-in-oil emulsions; solid and soft anhydrous gels; free and compacted powders, and two-phase forms. It may, for example, be in anhydrous form. The term “anhydrous composition” as used herein is defined above.

According to any one embodiment, the composition is in a form chosen from makeup compositions such as foundations, makeup rouges, eye-shadows, lipsticks, mascaras, and eyeliners; care compositions such as lipcare bases, care creams (day creams, night creams, anti-wrinkle creams and moisturizing creams), and makeup-removing creams and emulsions; and antisun compositions and self-tanning compositions; and haircare compositions such as care creams for the hair, the eyelashes and the eyebrows.

Uses

Disclosed herein is also the use of at least one fatty acid ester of dextrin with a degree of substitution of less than 2 on the basis of one repeating unit, for example one glucose unit, as described above, in a composition comprising a continuous liquid fatty phase, the fatty phase comprising at least one oil that has solubility parameters δd, δp and δh satisfying the following conditions:

[4(δd−δdₑ)²+(δp−δpₑ)²+(δh−δhₑ)²]^{1/2}≦10.

δdₑ, δpₑ and δhₑ being the solubility parameters of the at least one fatty acid ester of dextrin, to structure the composition and/or to limit the exudation of the composition, and/or to increase the gloss of the composition, and/or to limit the migration of the deposit of the composition, and/or to give the composition transparency.

Further disclosed herein is the use of at least one fatty acid ester of dextrin of formula (I) or (II) as described above, to increase the sheen of the composition and/or to improve the stability of the composition and/or to give the composition transparency; and/or to structure the fatty phase of the composition.

Further disclosed herein is, when the liquid fatty phase of the composition further comprises pigments and/or fillers, the use of at least one fatty acid ester of dextrin with a degree of substitution of the at least one fatty acid ester of dextrin of less than 2 on the basis of one repeating unit, for example one glucose unit, to improve the uniformity of the deposit of the composition, for example, its color uniformity.

Even further disclosed herein is the use of at least one fatty acid ester of dextrin with a degree of substitution of less than 2 on the basis of one repeating unit, for example, one glucose unit, and at least one fatty acid ester of dextrin with a degree of substitution of greater than 2 on the basis of one repeating unit, for example, one glucose unit, in a cosmetic composition comprising a continuous liquid fatty phase, to thicken or gel the composition and/or to increase the sheen of the composition and/or to limit the exudation of the composition, and/or to limit the migration of the composition, and/or to give the composition transparency and/or to obtain a stable composition and/or to obtain a deposit that is comfortable and/or uniform on a keratin material and/or to obtain a composition that applies easily to a keratin material.

Further disclosed herein is the use of a composition as defined above to obtain a deposit, for example, makeup, which is uniform and/or comfortable on a keratin material.

Further disclosed herein is the use of a sufficient amount of a mixture comprising at least one fatty acid ester of dextrin with a degree of substitution of less than 2 on the basis of one repeating unit, for example, one glucose unit, and at least one fatty acid ester of dextrin with a degree of substitution of greater than 2 on the basis of one repeating unit, for example, one glucose unit, in a composition comprising a liquid fatty phase and pigments, to improve the uniformity of the deposit of the composition, for example, its uniformity of color and/or to obtain a stable composition and/or to obtain a composition that applies easily to a keratin material.

Further disclosed herein is the use of at least one fatty acid ester of dextrin with a degree of substitution of less than 2 on the basis of one repeating unit, for example one glucose unit, as described above, in a wax-free anhydrous composition comprising a liquid fatty phase, to gel or thicken the said composition and/or to limit the exudation of the composition, and/or to increase the gloss of the composition, and/or to limit the migration of the deposit of the composition, and/or to give the composition transparency.

Further disclosed herein, when the liquid fatty phase of the wax-free anhydrous composition also comprises pigments and/or fillers, the use of at least one fatty acid ester of dextrin with a degree of substitution of the at least one fatty acid ester of dextrin of less than 2 on the basis of one repeating unit, for example, one glucose unit, to improve the uniformity of the deposit of the composition, for example, its color uniformity.

The embodiments disclosed herein are illustrated in greater detail in the examples that follow, without, however, being limiting in nature. The amounts are given as percentages by mass.

### EXAMPLE 1

10% polyethylene wax

5% dextrin palmitate Rheopearl TL (degree of substitution 1.5 to 1.7)

85% castor oil

It was noted that this composition was in the form of a stick that did not exude at 45° C. for 2 months.

### COMPARATIVE EXAMPLE 2

10% polyethylene wax

90% castor oil

This composition was in the form of a stick that exuded when it was placed under accelerated ageing conditions at 45° C. for 2 months.

### EXAMPLE 3

### Jar of gloss

| | | |
|---|---|---|
| Polydecene | | 21 |
| Octyldodecanol | | 30 |
| Tridecyl trimellitate | | 33.5 |
| Dextrin palmitate (Rhéopearl ® TL) | | 7.5 |
| Dextrin palmitate (Rhéopearl ® KL) | | 7.5 |
| Preserving agents | qs | 100 |

All the ingredients were mixed together with stirring at 90° C. until a uniform mixture was obtained. The mixture was poured into dishes and left to cool at room temperature.

The gloss had a transparent, shiny structure, and was easy to spread. It did not exude at 45° C. for 2 months.

### EXAMPLE 4

15% of dextrin palmitate Rheopearl KL (degree of substitution of 2.1 to 2.3)

15% of dextrin palmitate Rheopearl TL (degree of substitution 1.5 to 1.7)

70% of caprylic/capric acid triglyceride

It was noted that this composition was in the form of a stick that did not exude at 45° C. for 2 months.

### COMPARATIVE EXAMPLE 5

30% dextrin palmitate Rheopearl®KL (degree of substitution 2.1 to 2.3)

70% caprylic/capric acid triglyceride

This composition was in the form of a stick that exuded when it was placed under accelerated ageing conditions in an oven at 45° C. for 3 months.

### EXAMPLE 6

10% of Nikkol, dextrin palmitate, sold by the company Nikko Chemicals (totally esterified)

5% Rheopearl TL

85% caprylic/capric acid triglyceride oil

This composition was in the form of a stick that did not exude at 45° C. for 2 months.

### COMPARATIVE EXAMPLE 7

10% Nikkol, dextrin palmitate, sold by the company Nikko Chemicals (totally esterified)

90% caprylic/capric acid triglyceride oil

This composition was in the form of a stick that exuded, under accelerated ageing conditions at 45° C. for 2 months.

### EXAMPLE 8

An anhydrous foundation having the composition below was prepared:

| | | |
|---|---|---|
| Dextrin palmitate (Rheopearl ® TL) | | 5.3 |
| Dextrin palmitate (Rheopearl ® KL) | | 5.3 |
| Isododecane | qs | 100 |
| Isohexadecane | | 15 |
| Isononyl isononanoate | | 13.3 |
| Microcrystalline wax | | 3.2 |
| Polymethyl methacrylate | | 15 |
| Kaolin | | 3 |
| Talc | | 3 |
| Nylon-12 powder | | 8.5 |
| Hydrophobic coated iron oxides | | 1.8 |
| Hydrophobic coated titanium dioxide | | 6.7 |

The composition was prepared by mixing the microcrystalline wax, the isononyl isononanoate and the dextrin palmitates at 85° C.; next, without heating, the premilled mixture of pigments and of isohexadecane was added. The isododecane was then added at room temperature, followed by the fillers.

A foundation that was stable at 45° C. for two months was obtained. This foundation had a fondant texture when applied to the skin and formed a thin, non-sticky film. The makeup deposited on the skin showed good color uniformity.

### EXAMPLE 9

An anhydrous foundation having the composition below was prepared:

| | | |
|---|---|---|
| Dextrin palmitate (Rheopearl ® TLC) | | 4.8 |
| Dextrin palmitate (Rheopearl ® KL) | | 4.8 |
| Isododecane | qs | 100 |
| Isohexadecane | | 15 |
| Isononyl isononanoate | | 16.2 |
| Microcrystalline wax | | 3.2 |
| Polymethyl methacrylate | | 7.3 |
| Fumed silica | | 2.3 |
| Lauroyllysine | | 5 |
| Polyurethane-silica powder sold under | | 8 |
| the name Plastic Powder D-400 by | | |
| the company Toshiki) | | |
| Polymethylsilsesquioxane microbeads | | 5.2 |
| (Tospearl from Toshiba) | | |
| Hydrophobic coated iron oxides | | 2.1 |
| Hydrophobic coated titanium dioxide | | 6.3 |

The foundation showed good stability at 45° C. for 2 months. It applied easily to the face, giving a fondant effect, and formed a thin, non-sticky, uniform film.

What is claimed is:

1. A composition comprising a continuous liquid fatty phase gelled or thickened with a sufficient amount of at least one fatty acid ester of dextrin with a degree of substitution of less than 2 on the basis of one repeating unit, the fatty phase comprising at least one oil that has solubility parameters δd, δp and δh satisfying the following conditions:
[4(δd−δdₑ)²+(δp−δpₑ)²+(δh−δhₑ)²]^{1/2}≦10.
wherein δdₑ, δpₑ and δhₑ are the solubility parameters of the at least one fatty acid ester of dextrin, and wherein the liquid fatty phase and the at least one fatty acid ester of dextrin form a physiologically acceptable medium.

2. The composition according to claim 1, wherein the repeating unit of the at least one fatty acid ester of dextrin is a glucose unit.

3. A composition comprising a continuous liquid fatty phase gelled or thickened with a sufficient amount of a mixture comprising at least one fatty acid ester of dextrin with a degree of substitution of less than 2 on the basis of one repeating unit, and at least one fatty acid ester of dextrin with a degree of substitution of greater than 2 on the basis of one repeating unit, wherein the liquid fatty phase and the at least one fatty acid ester of dextrin form a physiologically acceptable medium.

4. The composition according to claim 3, wherein the repeating unit of the at least one fatty acid ester of dextrin is a glucose unit.

5. A wax-free anhydrous composition comprising a continuous liquid fatty phase thickened or gelled with a sufficient amount of at least one fatty acid ester of dextrin with a degree of substitution of less than 2 on the basis of one repeating unit to thicken or gel the liquid fatty phase, wherein the liquid fatty phase and the at least one fatty acid ester of dextrin form a physiologically acceptable medium.

6. The composition according to claim 5, wherein the repeating unit of the least one fatty acid ester of dextrin is a glucose unit.

7. The composition according to claim 1, wherein the at least one fatty acid ester of dextrin with a degree of substitution of less than 2 on the basis of a repeating unit, corresponds to formula (I): 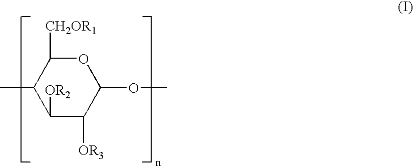
wherein:
the radicals R₁, R₂ and R₃, which may be identical or different, are each chosen from hydrogen and acyl groups R—CO— wherein the radical R is chosen from linear and branched, saturated and unsaturated hydrocarbon-based groups comprising from 6 to 50 carbon atoms, with the proviso that at least one of the radicals R₁, R₂ or R₃ is not a hydrogen atom, and
n is an integer ranging from 3 to 150.

8. The composition according to claim 7, wherein, in the definition of R, the hydrocarbon-based groups comprise from 8 to 30 carbon atoms.

9. The composition according to claim 8, wherein, in the definition of R, the hydrocarbon-based groups comprise from 12 to 22 carbon atoms.

10. The composition according to claim 9, wherein, in the definition of R, the hydrocarbon-based groups comprise from 12 to 18 carbon atoms.

11. The composition according to claim 7, wherein n is an integer ranging from 10 to 100.

12. The composition according to claim 11, wherein n is an integer ranging from 15 to 40.

13. The composition according to claim 1, wherein the at least one fatty acid ester of dextrin with a degree of substitution of less than 2 corresponds to formula (II): 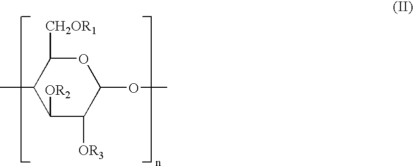
wherein:
radicals R₁, R₂ and R₃, which may be identical or different, are each chosen from hydrogen and acyl groups R—CO—, wherein the radical R is chosen from linear and branched, saturated and unsaturated hydrocarbon-based groups comprising from 6 to 50 carbon atoms, with the proviso that at least one of the radicals R₁, R₂ or R₃ is not a hydrogen, and
n is an integer ranging from 3 to 150.

14. The composition according to claim 13, wherein, in the definition of R, the hydrocarbon-based groups comprise from 8 to 30 carbon atoms.

15. The composition according to claim 14, wherein, in the definition of R, the hydrocarbon-based groups comprise from 12 to 22 carbon atoms.

16. The composition according to claim 15, wherein, in the definition of R, the hydrocarbon-based groups comprise from 12 to 18 carbon atoms.

17. The composition according to claim 16, wherein n is an integer ranging from 10 to 100.

18. The composition according to claim 17, wherein n is an integer ranging from 15 to 40.

19. The composition according to claim 3, wherein the at least one fatty acid ester of dextrin with a degree of substitution of greater than 2 corresponds to formula (III): 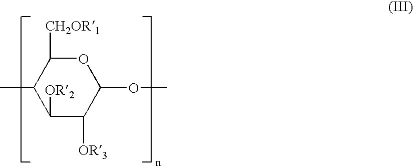
wherein:
radicals R′₁, R′₂ and R′₃, which may be identical or different, are each chosen from hydrogen and acyl groups R′—CO—, wherein the radical R′ is chosen from linear and branched, saturated and unsaturated hydrocarbon-based groups comprising from 6 to 50 carbon atoms, with the proviso that at least one of the radicals R′₁, R′₂ or R′₃ is not a hydrogen atom, and
n is an integer ranging from 3 to 150.

20. The composition according to claim 19, wherein, in the definition of R′, the hydrocarbon-based groups comprise from 8 to 30 carbon atoms.

21. The composition according to claim 20, wherein, in the definition of R′, the hydrocarbon-based groups comprise from 12 to 22 carbon atoms.

22. The composition according to claim 21, wherein, in the definition of R′, the hydrocarbon-based groups comprise from 12 to 18 carbon atoms.

23. The composition according to claim 19, wherein n is an integer ranging from 10 to 100.

24. The composition according to claim 23, wherein n is an integer ranging from 15 to 40.

25. The composition according to claim 7, wherein the R—CO— of formula (I) is chosen from at least one of caprylyl, caproyl, lauroyl, myristyl, palmityl, stearyl, eicosanyl, docosanoyl, isovaleryl, 2-ethylbutyryl, ethylmethylacetyl, isoheptanyl, 2-ethylhexanyl, isononanyl, isodecanyl, isotridecanyl, isomyristyl, isopalmityl, isostearyl, isohexanyl, decenyl, dodecenyl, tetradecenyl, myristyl, hexadecenoyl, palmitoleyl, oleyl, elaidyl, eicosenyl, sorbyl, linoleyl, linolenyl, punicyl, arachidonyl and stearoyl radicals.

26. The composition according to claim 13, wherein the R—CO— of formula (II) is chosen from at least one of caprylyl, caproyl, lauroyl, myristyl, palmityl, stearyl, eicosanyl, docosanoyl, isovaleryl, 2-ethylbutyryl, ethylmethylacetyl, isoheptanyl, 2-ethylhexanyl, isononanyl, isodecanyl, isotridecanyl, isomyristyl, isopalmityl, isostearyl, isohexanyl, decenyl, dodecenyl, tetradecenyl, myristyl, hexadecenoyl, palmitoleyl, oleyl, elaidyl, eicosenyl, sorbyl, linoleyl, linolenyl, punicyl, arachidonyl and stearoyl radicals.

27. The composition according to claim 19, wherein the R′—CO— of formula (III) is chosen from at least one of caprylyl, caproyl, lauroyl, myristyl, palmityl, stearyl, eicosanyl, docosanoyl, isovaleryl, 2-ethylbutyryl, ethylmethylacetyl, isoheptanyl, 2-ethylhexanyl, isononanyl, isodecanyl, isotridecanyl, isomyristyl, isopalmityl, isostearyl, isohexanyl, decenyl, dodecenyl, tetradecenyl, myristyl, hexadecenoyl, palmitoleyl, oleyl, elaidyl, eicosenyl, sorbyl, linoleyl, linolenyl, punicyl, arachidonyl and stearoyl radicals.

28. The composition according to claim 7, wherein the radical R of formula (I) is linear.

29. The composition according to claim 13, wherein the radical R of formula (II) is linear.

30. The composition according to claim 19, wherein the radical R′ of formula (III) is linear.

31. The composition according to claim 7, wherein the R—CO— of formula (I) is a palmitic radical.

32. The composition according to claim 13, wherein the R—CO— of formula (II) is a palmitic radical.

33. The composition according to claim 19, wherein the R′—CO— of formula (III) is a palmitic radical.

34. The composition according to claim 1, wherein the degree of substitution of the at least one fatty acid ester of dextrin with a degree of substitution less than 2 is less than 1.9.

35. The composition according to claim 34, wherein the degree of substitution is less than 1.8.

36. The composition according to claim 35, wherein the degree of substitution ranges from 1.5 to 1.7.

37. The composition according to claim 7, wherein the n of formula (I) ranges from 25 to 35.

38. The composition according to claim 37, wherein the n of formula (I) ranges from 27 to 33.

39. The composition according to claim 29, wherein the n of formula (I) is 30.

40. The composition according to claim 1, wherein the weight-average molecular weight of the at least one fatty acid ester of dextrin with a degree of substitution of less than 2 ranges from 10 000 to 30 000.

41. The composition according to claim 40, wherein the weight average molecular weight of the at least one fatty acid ester of dextrin with a degree of substitution of less than two ranges from 15 000 to 20 000.

42. The composition according to claim 3, wherein the at least one fatty acid ester of dextrin with a degree of substitution of greater than 2 has a degree of substitution greater than 2.1.

43. The composition according to claim 42, wherein the at least one fatty acid ester of dextrin with a degree of substitution of greater than 2 has a degree of substitution ranging from 2.1 to 2.3.

44. The composition according to claim 1, wherein the at least one fatty acid ester of dextrin with a degree of substitution of less than 2 on the basis of one repeating unit is present in an amount ranging from 1% to 50% by weight, relative to the total weight of the composition.

45. The composition according to claim 44, wherein the at least one fatty acid ester of dextrin with a degree of substitution of less than 2 on the basis of one repeating unit is present in an amount ranging from 4% to 30% by weight, relative to the total weight of the composition.

46. The composition according to claim 45, wherein the at least one fatty acid ester of dextrin with a degree of substitution of less than 2 on the basis of one repeating unit is present in an amount ranging from 4% to 25% by weight, relative to the total weight of the composition.

47. The composition according to claim 46, wherein the at least one fatty acid ester of dextrin with a degree of substitution less than 2 on the basis of one repeating unit is present in an amount ranging from 5% to 25% by weight, relative to the total weight of the composition.

48. The composition according to claim 47, wherein the at least one fatty acid ester of dextrin with a degree of substitution of less than 2 on the basis of one repeating unit is present in an amount ranging from 4% to 25% by weight, relative to the total weight of the composition.

49. The composition according to claim 48, wherein the at least one fatty acid ester of dextrin with a degree of substitution of less than 2 on the basis of one repeating unit is present in an amount ranging from 10% to 25% by weight, relative to the total weight of the composition.

50. The composition according to claim 3, wherein the mixture of the fatty acid esters of dextrin is present in an amount of less than or equal to 50%.

51. The composition according to claim 50, wherein the mixture of the fatty acid esters of dextrin is present in an amount ranging from 1% to 50% by weight, relative to the total weight of the composition.

52. The composition according to claim 51, wherein the mixture of the fatty acid esters of dextrin is present in an amount ranging from 5% to 40% by weight, relative to the total weight of the composition.

53. The composition according to claim 52, wherein the mixture of the fatty acid esters of dextrin is present in an amount ranging from 5% to 30% by weight, relative to the total weight of the composition.

54. The composition according to claim 56, wherein the mixture of the fatty acid esters of dextrin is present in an amount ranging from 9% to 25% by weight, relative to the total weight of the composition.

55. The composition according to claim 3, wherein the at least one fatty acid ester of dextrin with a degree of substitution of less than 2 and the at least one fatty acid ester of dextrin with a degree of substitution of greater than 2 are present in relative mass proportions ranging from 1:10 to 3:1.

56. The composition according to claim 55, wherein the at least one fatty acid ester of dextrin with a degree of substitution of less than 2 and the at least one fatty acid ester of dextrin with a degree of substitution of greater than 2 are present in relative mass proportions ranging from 1:2 to 3:2.

57. The composition according to claim 56, wherein the at least one fatty acid ester of dextrin with a degree of substitution of less than 2 and the at least one fatty acid ester of dextrin with a degree of substitution of greater than 2 are present in relative mass proportions ranging from 0.8:1 to 1.2:1.

58. The composition according to claim 57, wherein the at least one fatty acid ester of dextrin with a degree of substitution of less than 2 and the at least-one fatty acid ester of dextrin with a degree of substitution of greater than 2 are present in relative mass proportions ranging from 0.9:1 to 1.1:1.

59. The composition according to claim 58, wherein the at least one fatty acid ester of dextrin with a degree of substitution of less than 2 and the at least one fatty acid ester of dextrin with a degree of substitution of greater than 2 are present in relative mass proportions ranging from 1:2 to 1:1.

60. The composition according to claim 1, wherein the at least one oil is chosen from fatty acid triglycerides of 4 to 22 carbon atoms, hydroxylated triglycerides, synthetic alcohols comprising from 12 to 26 carbon atoms, and C₄ to C₂₂ fatty acid esters.

61. The composition according to claim 60, wherein the at least one oil is castor oil.

62. The composition according to claim 1, wherein the liquid fatty phase comprises at least one volatile organic oil.

63. The composition according to claim 62, wherein the at least one volatile organic oil is chosen from octamethylcyclotetrasiloxane, decamethylcyclopentasiloxane, dodecamethylcyclohexasiloxane, heptamethylhexyltrisiloxane, heptamethyloctyltrisiloxane, hexamethyld isiloxane, octamethyltrisiloxane, decamethyltetrasiloxane, dodecamethylpentasiloxane, isododecane, isodecane and isohexadecane.

64. The composition according to claim 63, wherein the at least one volatile organic oil is present in an amount ranging from 0.1% to 80% by weight, relative to the total weight of the composition.

65. The composition according to claim 64, wherein the at least one volatile organic oil is present in an amount-ranging from 1% to 60% by weight, relative to the total weight of the composition.

66. The composition according to claim 65, wherein the at least one volatile organic oil is present in an amount ranging from 5% to 50% by weight, relative to the total weight of the composition.

67. The composition according to claim 1, wherein the liquid fatty phase comprises at least one non-volatile oil.

68. The composition according to claim 67, wherein the at least one non-volatile oil is present in an amount ranging from 0.1% to 80% by weight, relative to the total weight of the composition.

69. The composition according to claim 68, wherein the at least one non-volatile oil is present in an amount ranging from 1% to 60% by weight, relative to the total weight of the composition.

70. The composition according to claim 69, wherein the at least one non-volatile oil is present in an amount ranging from 5% to 50% by weight, relative to the total weight of the composition.

71. The composition according to claim 1, wherein the liquid fatty phase comprises from 5% to 95% by weight of at least one oil, relative to the total weight of the composition.

72. The composition according to claim 71, wherein the liquid fatty phase comprises from 20% to 75% by weight of at least one oil, relative to the total weight of the composition.

73. The composition according to claim 72, wherein the liquid fatty phase comprises from 30% to 70% by weight: of at least one oil, relative to the total weight of the composition.

74. The composition according to claim 1, wherein
[4(δd−δdₑ)²+(δp−δpₑ)²+(δh−δhₑ)²]^{1/2}≦8.

75. The composition according to claim 1, wherein
[4(δd−δdₑ)²+(δp−δpₑ)²+(δh−δhₑ)²]^{1/2}≦5.

76. The composition according to claim 1, wherein the values of the Hansen solubility parameters for the at least one fatty acid ester of dextrin with a degree of substitution of less than 2 are such that 17≦δdₑ≦19, 1≦δpₑ≦2 and 9≦δhₑ≦11.

77. The composition according to claim 1, further comprising at least one additional non-waxy gelling or thickening system chosen from:
N-lauryl L-glutamate α,γ-di-N-butylamide,
monodibenzylidene sorbitol,
1,2- and 1,3-cyclohexane derivatives bearing at least one amide functional group, and
palmitates of dextrins and of fatty acid with a degree of substitution of greater than 2 relative to one glucose unit.

78. The composition according to claim 3, further comprising at least one additional non-waxy gelling or thickening system chosen from:
N-lauryl L-glutamate α,γ-di-N-butylamide,
monodibenzylidene sorbitol, and
1,2- and 1,3-cyclohexane derivatives bearing at least one amide functional group.

79. The composition according to claim 1, further comprising at least one dyestuff chosen from lipophilic dyes, hydrophilic dyes, pigments and nacres.

80. The composition according to claim 79, wherein the at least one dyestuff is present in an amount ranging from 0.1% to 50% by weight, relative to the total weight of the composition.

81. The composition according to claim 80, wherein the at least one dyestuff is present in an amount ranging from 5% to 25% by weight, relative to the total weight of the composition.

82. The composition according claim 1, wherein the composition is provided in solid or semi-solid forms.

83. The composition according to claim 1, wherein the composition is provided in the form of a rigid gel.

84. The composition according to claim 83, wherein the composition is provided in the form of a stick.

85. The composition according to claim 1, wherein the composition is provided in a form chosen from mascaras, eyeliners, foundations, lipsticks, blushes, deodorant products, makeup-removing products, makeup products for the body, eye-shadows, makeup rouges, concealer products, shampoos, conditioners, antisun compositions, and care products for the face and the body.

86. The composition according to claim 1, wherein the composition is provided in a form chosen from tubes of lipstick, lip balms, lip glosses, and compositions to be applied over a film of lipstick.

87. The composition according to claim 1, wherein the composition is free of waxes.

88. The composition according to claim 1, wherein the composition is anhydrous.

89. A method for increasing the gloss of a composition and/or improving the stability of a composition, comprising including at least one fatty acid ester of dextrin with a degree of substitution of less than 2 on the basis of one repeating unit, in a composition comprising a continuous liquid fatty phase, the fatty phase comprising at least one oil having solubility parameters δd, δp and δh satisfying the following conditions:
[4(δd−δdₑ)²+(δp−δpₑ)²+(δh−δhₑ)²]^{1/2}≦10.
wherein δdₑ, δpₑ and δhₑ are the solubility parameters of the dextrin ester, and wherein the least one fatty acid ester of dextrin with a degree of substitution of less than 2 on the basis of one repeating unit is effective for increasing the gloss of the composition and/or improving the stability of the composition.

90. The method according to claim 92, wherein the repeating unit of the at least one fatty acid ester of dextrin is a glucose unit.

91. A method of improving the uniformity of a deposit of a composition comprising including in the composition at least one fatty acid ester of dextrin with a degree of substitution of less than 2 on the basis of one repeating unit, in a composition comprising a continuous liquid fatty phase and pigments, wherein the at least one fatty acid ester of dextrin with a degree of substitution of less than 2 is effective for improving the uniformity of a deposit of the composition.

92. The method according to claim 91, wherein the uniformity of the deposit of the composition is color uniformity.

93. The method according to claim 91, wherein the repeating unit of the at least one fatty acid ester of dextrin is a glucose unit.

94. A method for imparting to a composition at least one property chosen from increasing the gloss of a composition, improving the stability of a composition, giving a composition transparency, gelling or thickening a fatty phase of a composition comprising including in the composition at least one fatty acid ester of dextrin with a degree of substitution of less than 2 on the basis of one repeating unit in a wax-free anhydrous composition comprising a liquid fatty phase, wherein the at least one fatty acid ester of dextrin with a degree of substitution of less than 2 is effective for imparting on the composition at least one of said properties.

95. The method according to claim 94, wherein the repeating unit of the at least one fatty acid ester of dextrin is a glucose unit.

96. A method for increasing the uniformity of a deposit of a composition comprising including in the composition at least one fatty acid ester of dextrin with a degree of substitution of less than 2 on the basis of one repeating unit, in a wax-free anhydrous composition comprising a liquid fatty phase and pigments, wherein the at least one fatty acid ester of dextrin with a degree of substitution of less than 2 on the basis of one repeating unit is effective for increasing the uniformity of the composition.

97. The method according to claim 96, wherein the repeating unit of the at least one fatty acid ester of dextrin is a glucose unit.

98. The method according to claim 97, wherein the uniformity of the deposit of the composition is color uniformity.

99. A method for imparting to a composition at least one property chosen from properties of increasing the sheen of a composition, improving the stability of a composition, limiting the exudation of a composition, giving a composition transparency, gelling or thickening a fatty phase of a composition, obtaining a stable composition, and obtaining a composition that applies easily to a keratin material comprising including in the composition a sufficient amount of a mixture comprising at least one fatty acid ester of dextrin with a degree of substitution of less than 2 on the basis of one repeating unit and at least one fatty acid ester of dextrin with a degree of substitution of greater than 2 on the basis of one repeating unit in a composition comprising a liquid fatty phase, wherein the mixture of fatty acid esters of dextrin is effective for imparting on the composition at least one of said properties.

100. The method according to claim 99, wherein the repeating unit of the at least one fatty acid esters of dextrin is a glucose unit.

101. A method for imparting on a composition at least one property chosen from properties of improving the uniformity of the deposit of a composition, obtaining a stable composition, and obtaining a composition that applies easily to a keratin material comprising including in the composition a sufficient amount of a mixture comprising at least one fatty acid ester of dextrin with a degree of substitution of less than 2 on the basis of one repeating unit and at least one fatty acid ester of dextrin with a degree of substitution of greater than 2 on the basis of one repeating unit in a composition comprising a liquid fatty phase and pigments, wherein the mixture of fatty acid esters of dextrin is effective for imparting on the composition at least one property.

102. The method according to claim 101, wherein the repeating unit of the at least one fatty acid esters of dextrin is a glucose unit.
